(19) United States
(12) Patent Application Publication
(10) Pub. No.: US 2017/0000630 A1
(43) Pub. Date: Jan. 5, 2017

(54) Universal Multi-Branch Endograft

(71) Applicants: Murray Shames, Tampa, FL (US); Gustavo Paludetto Oliveira, Brasilia (BR)

(72) Inventors: Murray Shames, Tampa, FL (US); Gustavo Paludetto Oliveira, Brasilia (BR)

(73) Assignee: UNIVERSITY OF SOUTH FLORIDA, TAMPA, FL (US)

(21) Appl. No.: 14/755,683

(22) Filed: Jun. 30, 2015

(51) Int. Cl. A61F 2/856; A61F 2/07; A61F 2/82

(52) U.S. Cl. CPC A61F 2/856; A61F 2/82; A61F 2/07; A61F 2002/065

(57) ABSTRACT

The present invention provides endovascular stent graft conversion devices, systems and related methods that are particularly useful in aortic aneurysm repair, such as, but not limited to, thoraco-abdominal aortic aneurysm (TAAA) repair. The endovascular stent graft conversion device provided by the present invention comprises a graft segment comprising a first end and a second end, wherein the first end is adapted for engaging a limb of an endograft device, and wherein the second end comprises three or more graft branches extending therefrom.

### BACKGROUND OF THE INVENTION

Surgeons regularly use customized, branched or fenestrated devices to treat elective thoraco-abdominal aortic aneurysm (TAAA). However, the device customization process is lengthy and necessitates a delay in treatment of sometimes more than a month. Further, the open repair of TAAAs commonly results in complications, particularly in patients with pre-existing comorbidities.

### BRIEF SUMMARY OF THE INVENTION

The present invention provides endovascular stent graft conversion devices, systems and related methods that are particularly useful in aortic aneurysm repair, such as, but not limited to, thoraco-abdominal aortic aneurysm (TAAA) repair. Such devices and systems are utilized with off-the-shelf components to enable surgeons to provide emergency endovascular treatment for TAAA without the necessity of customization of components.

In one aspect, the present invention provides an endovascular stent graft conversion device, comprising: a graft segment comprising a first end and a second end, wherein the first end is adapted for engaging a limb of an endograft device, and wherein the second end comprises three or more graft branches extending therefrom. In some embodiments, the first end is adapted for engaging the contralateral limb of a bifurcated endograft device.

In another aspect, the present invention provides a graft system useful for aneurysm repair, comprising: a bifurcated endovascular stent graft, wherein the endovascular stent graft comprises one or more limb; and a conversion device, the conversion device comprising a graft segment comprising a first end and a second end, wherein the first end is adapted for engaging one or more limb of the endovascular stent graft, wherein the second end comprises three or more graft branches extending therefrom. In some embodiments, the endovascular stent graft is a bifurcated stent graft. In some embodiments, the first end is adapted for engaging the contralateral limb of a bifurcated stent graft.

In some embodiments of the devices and systems of the present invention, the second end of the conversion device comprises three graft branches extending therefrom. In other embodiments, the second end comprises four graft branches extending therefrom.

In some embodiments of the devices and systems of the present invention, the first end of the graft segment of the conversion device is adapted for engaging a limb of an endovascular stent graft when the limb is in its deployed position. For example, the first end of the graft segment of the conversion device can be adapted for engaging the contralateral limb of a bifurcated endovascular stent graft when the contralateral limb is in its deployed position.

In another aspect, the present invention provides methods for thoraco-abdominal aortic aneurysm repair in a subject, comprising: providing a bifurcated endovascular stent graft, wherein the endovascular stent graft comprises a contralateral limb; deploying the endovascular stent graft within the aorta of the subject; providing a conversion device, the conversion device comprising a graft segment comprising a first end and a second end, wherein the first end is adapted for engaging the contralateral limb of the endovascular stent graft, wherein the second end comprises three or more graft branches extending therefrom; deploying the conversion device within the aorta of the subject; and engaging the contralateral limb of the endovascular stent graft with the first end of the conversion device.

In some embodiments, the methods further comprise engaging one or more visceral arteries, such as, for example, renal arteries, with one or more of the graft branches extending from the conversion device.

The devices, systems, and related methods herein described can be used in connection with pharmaceutical, medical, and veterinary applications, as well as fundamental scientific research and methodologies, as would be identifiable by a skilled person upon reading of the present disclosure. These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying figures in which:

FIG. 1 shows an embodiment of an endovascular stent graft conversion device of the present invention; and

FIG. 2 shows an embodiment of a graft system of the present invention comprising an endovascular stent graft and an endovascular stent graft conversion device, both in deployed positions prior to engagement with each other.

### DETAILED DESCRIPTION OF THE INVENTION

The present invention provides endovascular stent graft conversion devices, systems and related methods that are particularly useful in aortic aneurysm repair, such as, but not limited to, thoraco-abdominal aortic aneurysm (TAAA) repair.

Several aspects of the invention are described below. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including”, “includes”, “having”, “has”, “with”, or variants thereof, are intended to be inclusive similar to the term “comprising.”

As used herein, the term “subject” refers to an animal. Typically, the terms “subject” and “patient” may be used interchangeably herein in reference to a subject. As such, a “subject” includes a human that is being treated for a disease (e.g., aneurysm) as a patient.

The term “animal,” includes, but is not limited to, dog, cat, pig, monkey, chimpanzee, and human.

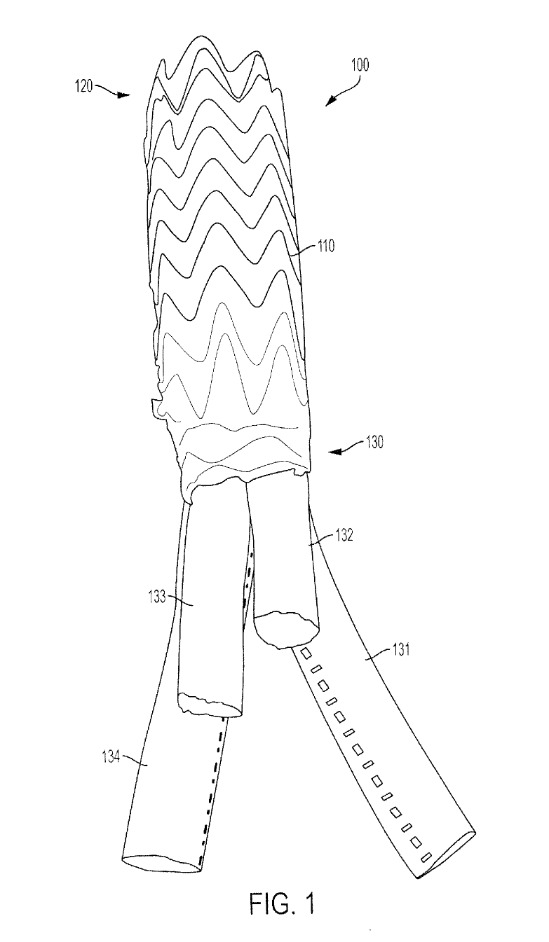

Referring to FIG. 1, the present invention provides an endovascular stent graft conversion device 100. The device 100 can include a graft segment 110 comprising a first end 120 and a second end 130, wherein the first end 120 is adapted for engaging a limb of an endograft device. The second end 130 comprises graft branches 131 extending therefrom. The embodiment shown utilizes four graft branches 131, 132, 133, 134; however, the device can utilize three, four, or more graft branches, depending on the intended utilization of the device. For example, it is useful to have four graft branches when using the device 100 for TAAA repair in order to provide branches for extending into the visceral and renal abranches of the surgical subject. For example, two of the branches are for engaging the superior mesenteric artery and the celiac artery, and two of the branches are for engaging the left and right renal arteries.

In some embodiments, the graft segment 110 of the endovascular stent graft conversion device 100 is about 16 mm in diameter. In some embodiments, the graft segment 110 measures about 3 cm in length as measured from the first end to the second end (not inclusive of the graft branches extending therefrom).

In some embodiments, the graft branches 131, 132, 133, 134 can be made of ePTFE (a strong, microporous material made from polytetrafluoroethylene) that are sutured, or otherwise attached, together in the depicted orientation. In some embodiments, the shorter branches (132, 133) are for engaging the superior mesenteric artery and the celiac artery, while the longer (131, 134) branches are for engaging the left and right renal arteries. The shorter branches can measure, e.g., 2 cm long and the longer branches can measure, e.g., 3 cm long. The shorter branches may measure, e.g., 7 mm in diameter and the longer branches may measure, e.g., 6 mm in diameter.

The shorter 7 mm branches are for the superior mesenteric artery and the celiac artery, while the longer 6 mm branches are for the left and right renal arteries.

In some embodiments, the first end 120 is adapted for engaging the contralateral limb of a bifurcated endograft device. The endovascular stent graft conversion device 100 allows the surgeon to utilize standard infrarenal aortic stent grafts; the first end can be adapted to engage a limb of any endograft device that is commercially available, providing a bridge device that facilitates treating complex aortic aneurysms without the need for preoperative customization of the individual components. Any of the currently available endograft devices (Gore® Excluder®, Gore® Excluder® C3, Medtronic® Endurant®, Medtronis® Endurant® II, or Cook® Zenith®) and future devices can be used with the conversion device of the present invention based on physician preference.

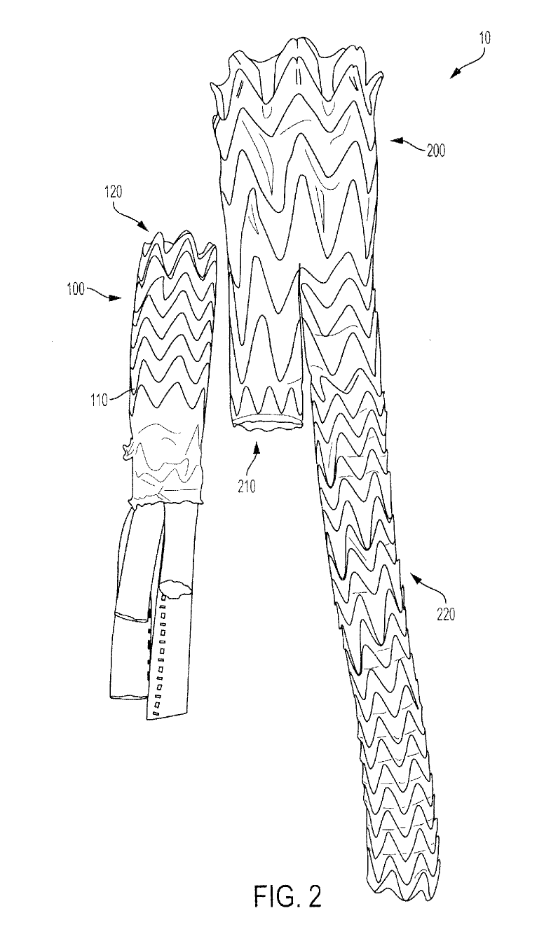

As shown in FIG. 2, the conversion device 100 of the present invention can also be utilized as a component of a graft system 10 utilized for aneurysm repair. The system 10 comprises an endovascular stent graft 200 with one or more limb 210, 220 and a stent graft conversion device 100, as described herein. As shown, the endovascular stent graft 200 can be a bifurcated stent graft. However, the endovascular stent graft 200 of the graft system 10 can alternatively utilize a single limb when treatment options do not necessitate a bifurcated system.

In some embodiments of the devices and systems of the present invention, the first end 120 of the graft segment 110 of the conversion device 100 is adapted for engaging a limb of an endovascular stent graft when the limb is in its deployed position. For example, the first end 120 of the graft segment 110 of the conversion device 100 can be adapted for engaging the contralateral limb (i.e., 210) of a bifurcated endovascular stent graft when the contralateral limb is in its deployed position, as shown in FIG. 2.

In another aspect, the present invention provides methods for TAAA repair in a subject, comprising: providing a bifurcated endovascular stent graft, wherein the endovascular stent graft comprises a contralateral limb; deploying the endovascular stent graft within the aorta of the subject; providing a conversion device, the conversion device comprising a graft segment comprising a first end and a second end, wherein the first end is adapted for engaging the contralateral limb of the endovascular stent graft, wherein the second end comprises three or more graft branches extending therefrom; deploying the conversion device within the aorta of the subject; and engaging the contralateral limb of the endovascular stent graft with the first end of the conversion device.

In some embodiments, each limb of the stent graft will be pre-wired with guidewire and delivered into the contralateral gate of the bifurcated graft that is placed into the thoracic aorta. In some embodiments, 0.014 inch guidewire is utilized.

The endovascular aortic repair device can be extended proximally in the thoracic aorta by using a standard commercially available thoracic endograft (TEVAR). Once the bifurcated device is secured in the thoracic aorta with the contralateral limb above, e.g., about 3-4 cm above, the celiac artery the contralateral gate can be cannulated from the femoral access site. The conversion device is then delivered into the contralateral limb and deployed.

In some embodiments, from the axillary access site, each of a 7-12 F sheath is advanced to the proximal aspect of the conversion device. Using an endovascular snare, the wire through the celiac limb is captured and pulled through the axillary sheath obtaining through access through the “branch”. Using standard endovascular techniques, the target artery is catheterized and the wire and sheath advanced into the artery.

An appropriately sized stent graft (e.g., Gore® Viabahn® or Atrium® iCAst®) is advanced through the sheath to bridge the target artery into the conversion limb branch. The stent graft is deployed and balloon dilated (as necessary). In preferred embodiments, a minimum of about 2 cm of overlap is recommended into the conversion branch limb. This process can be repeated up to 4 times (for each branch) until all visceral target arteries are bridged into the conversion device.

This part of the procedure is completed by extending the ipsilateral limb of the bifurcated endograft into health distal aorta either with a limb extension or second bifurcated endovascular stent grafts.

In some embodiments, the methods further comprise engaging one or more visceral arteries, such as, for example, renal arteries, with one or more of the graft branches extending from the conversion device.

It is to be appreciated that the foregoing Detailed Description section, and not the

Abstract section, is intended to be used to interpret the claims. The Abstract section may set forth one or more, but not all, exemplary embodiments of the present invention as contemplated by the inventor(s), and thus, is not intended to limit the present invention and the appended claims in any way.

The foregoing description of the specific embodiments should fully reveal the general nature of the invention so that others can, by applying knowledge within the skill of the art, readily modify and/or adapt for various applications such specific embodiments, without undue experimentation, without departing from the general concept of the present invention. Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents. Moreover, the breadth and scope of the present invention should not be limited by any of the above-described exemplary embodiments, but should similarly be defined only in accordance with the following claims and their equivalents.

1. An endovascular stent graft conversion device, comprising:
a graft segment comprising a first end and a second end, wherein the first end is adapted for engaging a limb of an endograft device, wherein the second end comprises three or more graft branches extending therefrom, wherein the three or more graft branches all extend from the second end at the same location.

2. The device of claim 1, wherein the second end comprises three graft branches extending therefrom.

3. The device of claim 1, wherein the second end comprises four graft branches extending therefrom.

4. The device of claim 1, wherein the endograft device comprises a first limb and a contralateral limb and wherein the first end of the endovascular stent graft conversion device is adapted for engaging the contralateral limb of the endograft device.

5. A graft system, comprising:
an endovascular stent graft, wherein the endovascular stent graft comprises one or more limb; and
a conversion device, the conversion device comprising a graft segment comprising a first end and a second end, wherein the first end is adapted for engaging one of the limbs of the endovascular stent graft, wherein the second end comprises three or more graft branches extending therefrom.

6. The system of claim 5, wherein the second end comprises three graft branches extending therefrom.

7. The system of claim 5, wherein the second end comprises four graft branches extending therefrom.

8. The system of claim 5, wherein the endovascular stent graft is a bifurcated stent graft with two limbs.

9. The system of claim 5, wherein the first end of the graft segment of the conversion device is adapted for engaging one of the limbs of the endovascular stent graft when the limb is in its deployed position.

10. A method for thoraco-abdominal aortic aneurysm repair in a subject, comprising:
providing a bifurcated endovascular stent graft, wherein the endovascular stent graft comprises a contralateral limb;
deploying the endovascular stent graft within the aorta of the subject;
providing a conversion device, the conversion device comprising a graft segment comprising a first end and a second end, wherein the first end is adapted for engaging the contralateral limb of the endovascular stent graft, wherein the second end comprises three or more graft branches extending therefrom;
deploying the conversion device within the aorta of the subject; and
engaging the contralateral limb of the endovascular stent graft with the first end of the conversion device.

11. The method of claim 10, further comprising engaging one or more visceral arteries with one or more of the graft branches extending from the conversion device.

12. The method of claim 11, wherein the one or more visceral arteries include renal arteries.

13. The device of claim 1, wherein the second end has a terminal aperture at a terminus of the second end, and wherein the three or more graft branches all extend from the terminal aperture.

14. The device of claim 1, wherein the three or more graft branches extending from the second end are adapted to extend into the visceral and/or renal branches of an aorta.
